(19) United States
(12) Patent Application Publication
(10) Pub. No.: US 2025/0319226 A1
(43) Pub. Date: Oct. 16, 2025

(54) A DISINFECTANT FOGGING AND FILTERING DEVICE

(71) Applicant: SQUIRE KENT PTY LIMITED, Crowsnest (AU)

(72) Inventor: Peter YASSA, Bella Vista (AU)

(21) Appl. No.: 18/865,237

(22) PCT Filed: May 11, 2023
(86) PCT No.: PCT/AU2023/050393

§ 371 Date: Nov. 12, 2024

(30) Foreign Application Priority Data
May 15, 2022 (AU) 2022901295

(51) Int. Cl. A61L 9/12; B01D 46/00; B01D 46/58

(52) U.S. Cl. CPC A61L 9/122; B01D 46/0005; B01D 46/0028; B01D 46/0047; B01D 46/58; A61L 2209/11; A61L 2209/12; A61L 2209/133; A61L 2209/134; A61L 2209/14; A61L 2209/15; B01D 2273/30; B01D 2279/50; B01D 2279/65

(57) ABSTRACT

Disclosed herein is a disinfectant fogger and filtration device for use in an enclosed space. The device may comprise; a fogger unit comprising: a container containing a disinfectant solution; a solution pump fluidically connected to the container for pumping the solution out of the container; a fogger fan fluidically connected to the pump and solution for discharging the solution out of the fogger unit; a first air channel fluidically connected to the fogger fan, the first air channel comprising a first opening to allow air from the enclosed space to enter the fogger unit, and a second opening adjacent the fogger fan to allow air to enter the fan and mix with the solution. The device may also comprise a filtration unit mounted to the fogger unit, the filtration unit comprising: a first filter disposed adjacent a first opening disposed in a wall of the filtration unit; a filtration fan fluidically connected to the filter for extracting air from outside the filtration unit though the filter to filter the solution from the air, the filtration fan being configured to return the filtered air to the enclosed space.

### TECHNICAL FIELD

The present disclosure relates to a disinfecting fogging and filtering device for disinfecting an enclosed space.

### BACKGROUND ART

Microbial contamination of enclosed spaces (e.g. a house, an office, a room in an airport etc) may be a source of infection and diseases for humans. A recent example includes viruses that cause the ongoing COVID-19 pandemic. Microbes adhere to surfaces of an enclosed space and also remain airborne within an enclosed space after being discharged by, for example, a person coughing or sneezing. Reducing or eliminating harmful microbes in an enclosed space is a problem.

The most common method to clean enclosed spaces is manual sterilisation. For example, cleaners spend a considerable amount of time cleaning and disinfecting an enclosed space. Another method for disinfecting airborne microbes within an enclosed space is to use disinfectant fogging devices. Disinfectant fogging devices may include a container than includes a disinfectant solution and a means for discharging the solution, typically vertically, to the enclosed space. The disinfectant solution may be discharged as an aerosol mist to disinfect the enclosed space. An issue with existing fogging devices is that the enclosed space may not be used for a lengthy period of time (e.g. 24 hours) as the aerosol mist discharged into the space may be harmful to humans.

In this specification, unless the contrary is expressly stated, where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

### SUMMARY

Disclosed herein is a disinfectant fogger and filtration device for use in an enclosed space. The device may comprise; a fogger unit comprising: a container containing a disinfectant solution; a solution pump fluidically connected to the container for pumping the solution out of the container; a fogger fan fluidically connected to the pump and solution for discharging the solution out of the fogger unit; a first air channel fluidically connected to the fogger fan, the first air channel comprising a first opening to allow air from the enclosed space to enter the fogger unit, and a second opening adjacent the fogger fan to allow air to enter the fan and mix with the solution; a filtration unit mounted to the fogger unit, the filtration unit comprising: a first filter disposed adjacent a first opening disposed in a wall of the filtration unit; a filtration fan fluidically connected to the filter for extracting air from outside the filtration unit though the filter to filter the solution from the air, the filtration fan being configured to return the filtered air to the enclosed space.

In some forms, the fogger unit comprises a fogger housing for supporting the container solution pump and the fogger fan.

In some forms, the first air channel is formed from a cavity disposed within the fogger housing.

In some forms, the first opening of the first air channel is disposed in a rear wall of the fogger housing, and the first air channel extends from the first opening along a longitudinal axis of the fogger unit.

In some forms, the fogger fan extends along a second axis of the fogger unit, the second axis being substantially perpendicular to the longitudinal axis, and wherein the solution is discharged through a top wall of the fogger housing.

In some forms, the fogger unit may comprise a solution channel disposed between the container and the fogger fan, the solution channel being offset from and substantially parallel to the first air channel.

In some forms, the container is user replaceable.

In some forms, the fogger unit may comprise a controller for controlling the operation of the fogger fan and solution pump.

In some forms, the controller is a programable logic controller.

In some forms, fogger unit may comprise a status indicator light connected to the controller, the status indicator light being configured to alert a user when the device is in operation.

In some forms, the filtration unit may comprise a filtration housing for supporting the first filter and the filtration fan.

In some forms, the filtration housing may comprise top, side, front, rear and underside walls, the side walls being substantially perpendicular to the top, rear and front walls.

In some forms, the underside wall comprises first and second connected portions, the first portion being disposed towards the front wall and substantially parallel to the top wall, the second potion being disposed towards the rear wall and forming an acute angle with the first portion and rear wall.

In some forms, the filtration unit may comprise wheels that are mounted to the second portion of the underside wall and feet that are mounted to and extend away from the first potion of the underside wall, the wheels and feet together supporting the filtration unit off a ground surface of the encloses space in use.

In some forms, the side walls of the filtration housing comprise first and second spaced side walls, the first side wall comprising the first air opening, the second side wall comprising a second air opening.

In some forms, the filtration device may comprise a second filter disposed adjacent the second opening.

In some forms, the first and second filters are disposed outside the filter housing.

In some forms, the filter unit may comprise first and second pockets mounted to the filter housing, the first pocket being configured to support the first filter and the second pocket being configured to support the second filter.

In some forms, the filter housing may comprise first and second mounting rails disposed on the top wall of the filter housing, the mounting rails being configured to support the fogger unit.

In some forms, the first and second mounting rails are spaced from one another such that the fogger unit is able to be mounted between the mounting rails.

In some forms, the filter housing may comprise an air outlet disposed in the front wall of the filter housing, the air outlet being fluidically connected to the filtration fan.

In some forms, the filtration unit may comprise a handle mounted to the rear wall of the filtration housing.

In some forms, the handle extends above the fogger unit in use and is configured to enable the device to be moved.

In some forms, the filtration unit may comprise first and second carry handles, the first carry handle being mounted to the front wall of the filtration housing, and the second handle being mounted to the rear wall of the filtration housing, together the first and second carry handles being configured to enable the device to be lifted by users.

In some forms, the filtration fan is a squirrel cage fan.

In some forms, the fogger unit may comprise a tundish for collecting fluid that is not discharged from the fogger unit in use.

### BRIEF DESCRIPTION OF DRAWINGS

Various embodiments/aspects of the disclosure will now be described with reference to the following figures.

FIG. 1 shows a side view of the disinfectant fogger and filtration device according to the present disclosure;

FIG. 2 shows a cross-sectional view through the device shown in FIG. 1;

FIG. 3 shows a perspective view of the device shown in FIG. 1 with the fogger unit detached from the filtration unit;

FIG. 4 shows a rear view of the device shown in FIG. 1;

FIG. 5 shows a top view of the device shown in FIG. 1;

FIG. 6 shows a perspective view of the device shown in FIG. 1 with the filters removed; and

FIG. 7 shows another side view of the device shown in FIG. 1.

### DETAILED DESCRIPTION

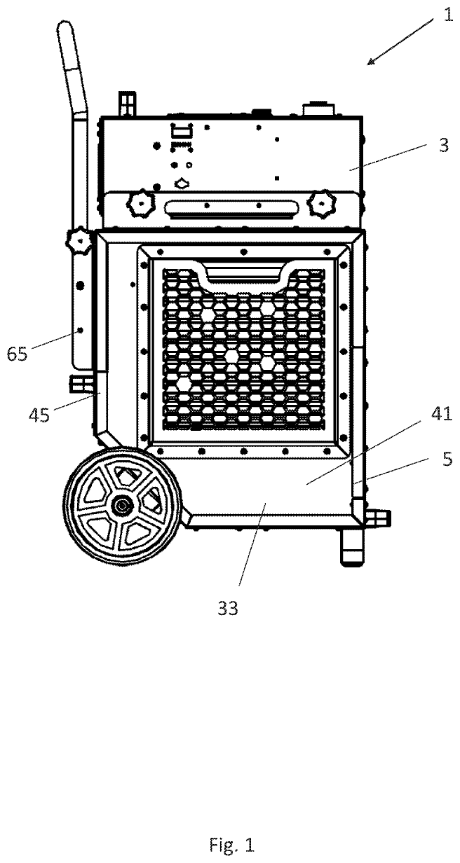

Disclosed herein is a disinfectant fogger and filtration device for use in an enclosed space. The disinfectant fogger and filtration device will be described with reference to FIGS. 1 to 7.

FIG. 1 shows a side view of the disinfectant fogger and filtration device 1. The device 1 includes two primary components, being a fogger unit 3 and a filtration unit 5. Together, the fogger 3 and filtration 5 units enable an enclosed space to be disinfected and filtered simultaneously, or disinfected and then filtered sequentially. Advantageously, the disclosed disinfectant fogger and filtration device 1 enables the enclosed space to be treated and then cleared by a single piece of equipment. This significantly shortens the amount of time that a room is unable to be inhabited (e.g. from 24 hours down to one cycle, which may be as short as 15 minutes depending on the room size).

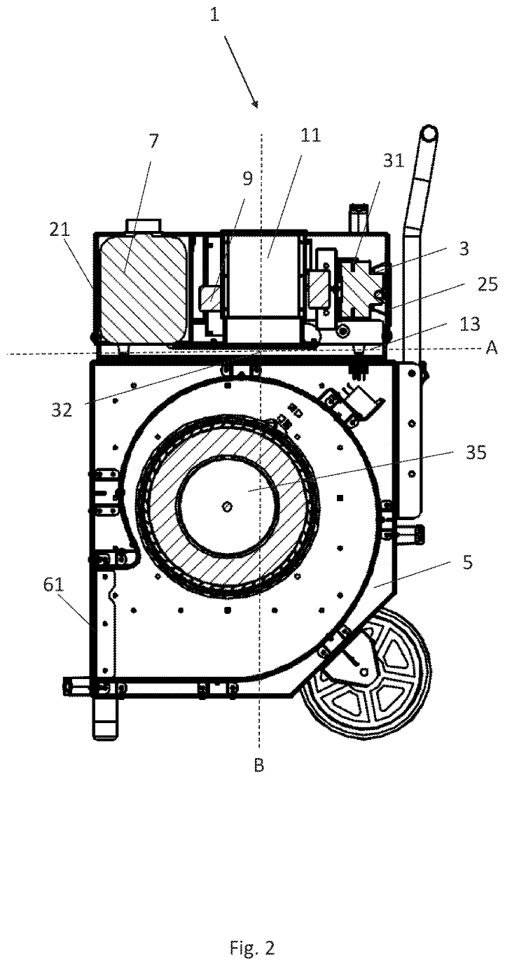

The fogger and filtration units will now be described in further detail with reference to FIG. 2, which shows a cross-sectional view through the device 1. The fogger unit 3 includes a container 7 containing a disinfectant solution. The container 7 is in the form of a bottle that is able to be accessed and replaced by a user of the device (e.g. to replace the bottle or to re-fill the bottle as required). As will be appreciated by the skilled addressee, the bottle may contain several types of fluids for fogging purposes, including fluids that serve as an effective disinfectant in fog form. In the detailed embodiment, the fluid in the bottle is not stored under pressure and has no propellant. In other embodiments, the fluid in the bottle may be stored under pressure such that it is vapourised upon release from the bottle.

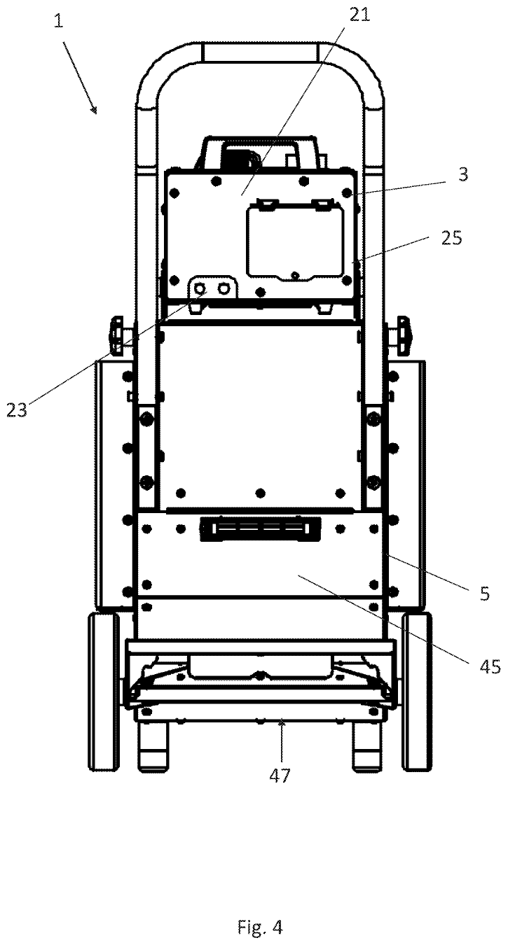

The fogger unit 3 includes a solution pump 9 fluidically connected to the container 7 for pumping the solution out of the container to a fogger fan 11. The fan 11 is fluidically connected to the pump 9 and solution for discharging the solution out of the fogger unit 3. The fogger unit includes a first air channel 13 fluidically connected to the fogger fan 11, the first air channel including a first opening (see FIG. 4) to allow air from the enclosed space to enter the fogger unit 3, and a second opening (not shown) adjacent the fogger fan 11 to allow air to enter the fan 11 and mix with the vaporised disinfectant solution.

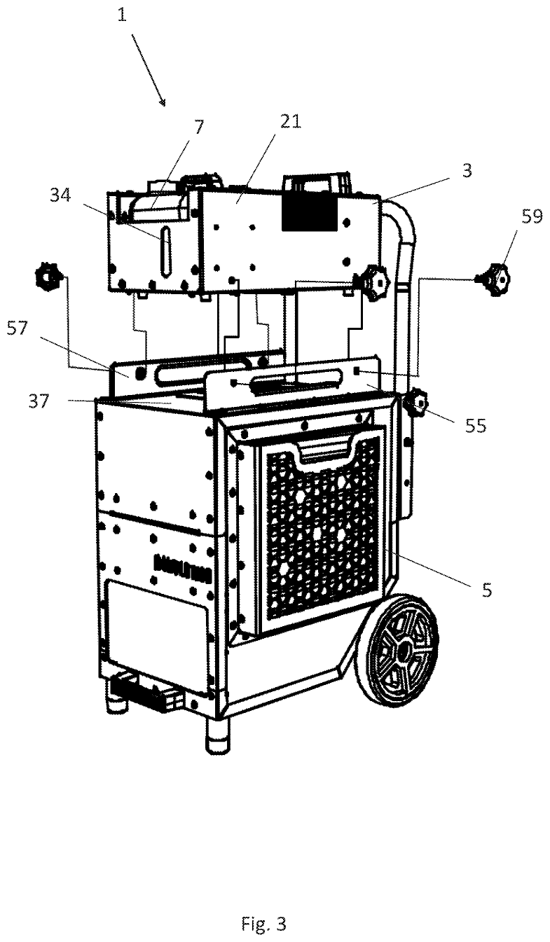

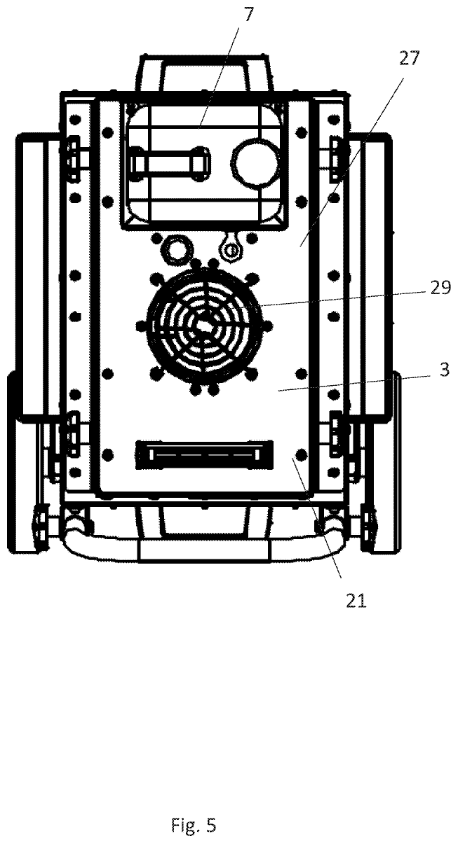

The fogger unit will now be described with reference to FIGS. 2-5. FIG. 3 shows a perspective view of the device 1 with the fogger unit 3 detached from the filtration unit 5. FIG. 4 shows a rear view of the device 1. FIG. 5 shows a top view of the device 1. The fogger unit includes a fogger housing 21 for supporting the container 7, solution pump 9 and the fogger fan 11. The first air channel 13 is formed from a cavity disposed within the fogger housing 21. The first opening 23 of the first air channel is disposed in a rear wall 25 of the fogger housing, and the first air channel 13 extends from the first opening 23 along a longitudinal axis A of the fogger unit 3.

The fogger fan 11 extends along a second axis B of the fogger unit. The second axis B is substantially perpendicular to the longitudinal axis A (see FIG. 2). Solution from the container 7 is discharged through a top wall 27 of the fogger housing 21 (see FIG. 5) via an air vent 29 (i.e. vertically) that is aligned with the second axis B. The fogger unit 3 includes a solution channel (not shown) disposed between the container 7 and the fogger fan 11. In the detailed embodiment, the solution channel is offset from and substantially parallel to the first air channel 13 (and longitudinal axis A). The housing 21 includes an aperture 34 formed therethrough that is aligned with the container to enable a user to check the solution level without having to remove the container 7.

The fogger unit 3 includes a controller for controlling the operation of the fogger fan 11 and solution pump 9. In the detailed embodiment, the controller is a programable logic controller 31 that is mounted within the fogger housing 21 of the fogger unit 3. The controller 31 is configured to operate the electronic components of the fogger unit 3 and filtration unit 5. As will be appreciated by the skilled addressee, the controller 31 may be configured with several user selectable operations. In the detailed embodiment, the controller 31 includes a wireless connection with a remote that enables the user to safely operate the device 1 from outside the enclosed space. The fogger unit also a status indicator light connected to the controller 31 that is configured to alert a user when the device 1 is in operation.

The fogger unit 3 also includes a tundish 32 disposed below the fan 11 for collecting fluid that is not discharged from the fogger unit 3 in use.

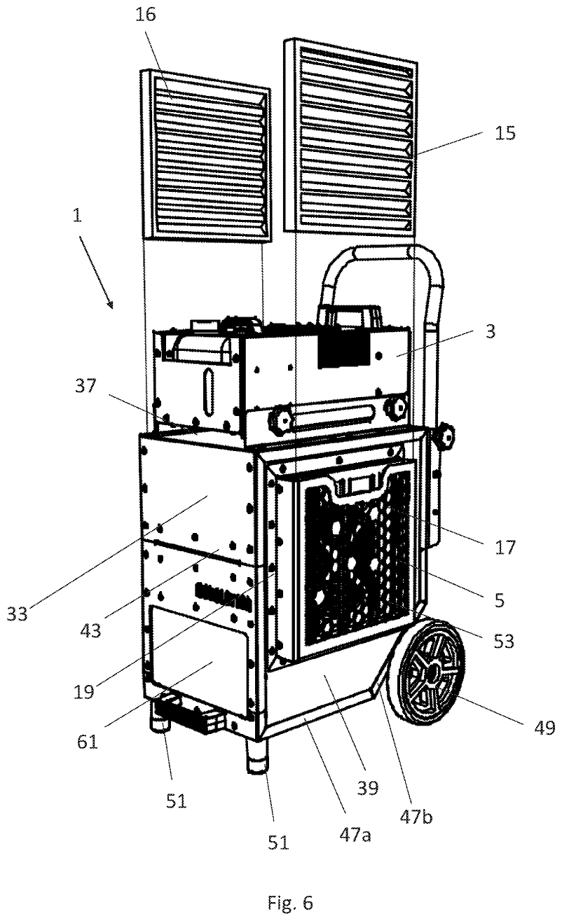

The filtration unit 5 is mounted to the fogger unit 3. In the detailed embodiment, the filtration unit 5 is positioned below the fogger unit 3 because the device delivers fog that initially rises, and the filter and fan extracts the fog as it settles. This creates a vortex effect. The filtration unit will now be described with reference to FIGS. 1 to 6. FIG. 6 shows a perspective view of the device 1 with the filters 15, 16 removed.

The filtration unit 5 includes a filtration housing 33 for supporting the filters 15, 16 and a filtration fan 35. In the detailed embodiment, the housing 33 is formed from durable power-coated sheet metal. The filtration housing 33 includes top 37, left side 39, right side 41 (see FIG. 1), front 43, rear 45 (see FIG. 4) and underside 47 (see FIG. 4) walls. The side walls 39, 41 are substantially perpendicular to the top 37, rear 45 and front 43 walls. The underside wall includes first 47a and second 47b connected portions (see FIG. 6). The first portion 47a is disposed towards the front wall 43 and is substantially parallel to the top wall 37. The second potion 47b extends from the first portion 47a is disposed towards the rear wall 45, and forms an acute angle with the first portion 47a and rear wall 45. In other words, the second portion 47b extends along a plane that forms an acute angle (approximately 45° in the detailed embodiment) with planes that extends along both the rear wall 45 and the first portion 47a of the underside wall.

The filtration unit 5 includes two wheels 49 that are mounted to the second portion 47b of the underside wall. The filtration unit 5 also includes feet 51 that are mounted to and extend away from the first potion 47a of the underside wall. Together, the wheels 49 and feet 51 support the filtration unit 5 off a ground surface of the enclosed space in use (i.e. there is a gap between the underside of the filtration unit 5 and the ground). In the detailed embodiment, the wheels are large in diameter to assist with climbing stairs and uneven surfaces. The wheels are also non-marking so as not to damage surfaces of the enclosed space.

As is shown in FIG. 2, the filtration unit 5 includes a filtration fan 35 fluidically connected to the filters 15, 16. The filtration fan 35 is configured to extract air from outside the filtration unit 5, though the filters 15, 16, to filter the solution from the air. The fan 35 then returns the filtered air to the enclosed space. In the detailed embodiment, the filtration fan 35 is a squirrel cage fan.

The side walls 39, 41 of the filtration housing 33 are spaced from one another, with the fan 35 being housed therebetween (i.e. within a cavity of the housing 33). The left side wall 39 includes a first air opening 17. The second side wall 41 includes a similar (second) air opening (not shown). The air openings in the side walls 39, 41 of the housing 33 allow air to be extracted by the fan 35 from the enclosed space into the housing 33. In the detailed embodiment, louvres or grills are disposed within the air openings in the side walls 39, 41.

The filtration device 5 includes two filters 15, 16. The filter dimensions, material and other specifications may be selected by a user to suit the required performance of the filtration unit 5. The filters are disposed adjacent the first and second air openings of the side walls 39, 41. Both are disposed outside of the housing 33 such that they are readily accessible by a user for replacement and maintenance purposes. The filter unit 5 includes first 53 and second (not shown) pockets mounted to the outside of the filter housing 33 that are configured to support the filters adjacent the air openings in the side walls 39, 41. In the detailed embodiment, the pockets are formed from plastic material and are perforated to allow air to enter the pockets and filter housed therein.

In use, the fan 35 extracts air from the encloses space through the filters 15, 16 to filter and clean the air (e.g. to remove the solution dispensed by the fogging device). The fan 35 then returns the filtered air to the enclosed space.

As is shown in FIG. 3, the filter housing also includes first 55 and second 57 mounting rails disposed on the top wall 37 of the filter housing 33. The mounting rails are configured to support the fogger unit 3. The mounting rails 55, 57 are spaced from one another such that the fogger unit 3 is able to be mounted between the mounting rails 55, 57. The mounting rails 55, 57 and the fogger housing 21 include a plurality of corresponding mounting apertures that are configured to receive a plurality of fasteners 59 (four in the detailed embodiment).

As is shown in FIGS. 2 and 6, the filter housing 33 includes an air outlet 61 disposed in the front wall 43 of the filter housing 33. The air outlet 61 is fluidically connected to the filtration fan 35 such that the fan is able to return the filtered air to the enclosed space via the outlet 61. In the detailed embodiment, a grill/mesh is positioned over the air outlet 61 with small perforation to prevent injury to a user (e.g. to prevent a user from inserting a finger into the fan).

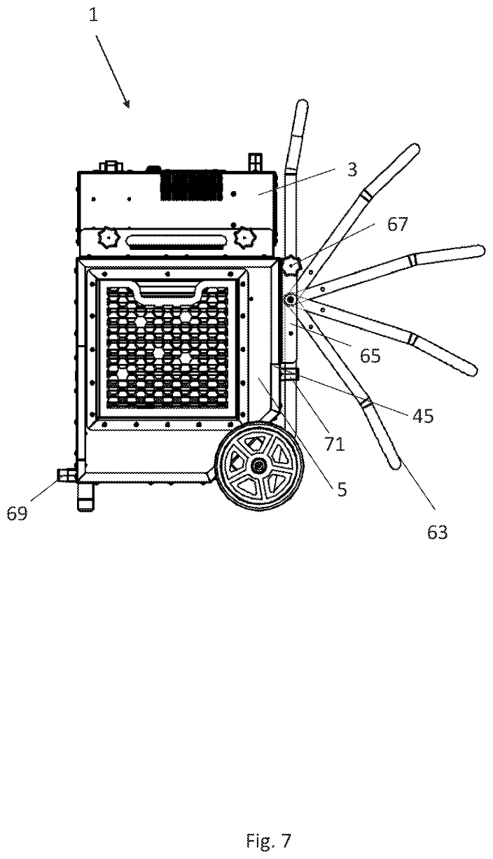

As is shown in FIG. 7, which provides another side view of the device 1, the filtration unit 5 includes a handle 63 that is pivotally mounted to the rear wall 45 of the filtration housing 33. Two spaced mounting plates 65 (see also FIG. 1) extend from the rear wall 45 of the filtration housing 33. The handle 63 is mounted between the mounting plates 65. The handle 63 and mounting plates 65 include corresponding mounting apertures to lock the handle in position. For example, the handle 63 is able to be locked in an upright position using fasteners 67 such that it extends above the fogger unit 5 in use to enable the device 1 to be readily moved into, and within, the enclosed space.

The filtration unit also includes first 69 and second 71 carry handles. The first carry handle 69 is mounted to the front wall of the filtration housing. The second handle 71 is mounted to the rear wall of the filtration housing. Together the first and second carry handles are configured to enable the device to be lifted by users.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

1. A disinfectant fogger and filtration device for use in an enclosed space, the device comprising:
a fogger unit comprising:
a container containing a disinfectant solution,
a solution pump fluidically connected to the container for pumping the solution out of the container,
a fogger fan fluidically connected to the pump and solution, and
a first air channel fluidically connected to the fogger fan, the first air channel comprising a first opening to allow air from the enclosed space to enter the fogger unit, and a second opening adjacent the fogger fan to allow air to enter the fan and mix with the solution, the fogger fan being configured to discharge the solution out of the fogger unit and into the enclosed space as a vaporised disinfectant solution;
and a filtration unit mounted to the fogger unit, the filtration unit comprising:
a first filter disposed adjacent a first opening disposed in a wall of the filtration unit, and
a filtration fan fluidically connected to the filter for extracting air from outside the filtration unit through the filter to filter the disinfectant solution from the air, the filtration fan being configured to return the filtered air to the enclosed space.

2. A disinfectant fogger and filtration device according to claim 1, the fogger unit comprising a fogger housing for supporting the container solution pump and the fogger fan.

3. A disinfectant fogger and filtration device according to claim 2, wherein the first air channel is formed from a cavity disposed within the fogger housing.

4. A disinfectant fogger and filtration device according to claim 3, wherein the first opening of the first air channel is disposed in a rear wall of the fogger housing, and the first air channel extends from the first opening along a longitudinal axis of the fogger unit.

5. A disinfectant fogger and filtration device according to claim 4, wherein the fogger fan extends along a second axis of the fogger unit, the second axis being substantially perpendicular to the longitudinal axis, and wherein the solution is discharged through a top wall of the fogger housing.

6. A disinfectant fogger and filtration device according to claim 5, wherein the fogger unit comprises a solution channel disposed between the container and the fogger fan, the solution channel being offset from and substantially parallel to the first air channel.

7. A disinfectant fogger and filtration device according to claim 1, wherein the container is user replaceable.

8. A disinfectant fogger and filtration device according to claim 1, wherein the fogger unit comprises a controller for controlling the operation of the fogger fan and solution pump.

9. A disinfectant fogger and filtration device according to claim 8, wherein the controller is a programable logic controller.

10. A disinfectant fogger and filtration device according to claim 1, wherein the fogger unit comprises a status indicator light connected to the controller, the status indicator light being configured to alert a user when the device is in operation.

11. A disinfectant fogger and filtration device according to claim 1, the filtration unit comprising a filtration housing for supporting the first filter and the filtration fan.

12. A disinfectant fogger and filtration device according to claim 11, wherein the filtration housing comprises top, side, front, rear and underside walls, the side walls being substantially perpendicular to the top, rear and front walls.

13. A disinfectant fogger and filtration device according to claim 12, wherein the underside wall comprises first and second connected portions, the first portion being disposed towards the front wall and substantially parallel to the top wall, the second portion being disposed towards the rear wall and forming an acute angle with the first portion and rear wall.

14. A disinfectant fogger and filtration device according to claim 13, wherein the filtration unit comprises wheels that are mounted to the second portion of the underside wall and feet that are mounted to and extend away from the first portion of the underside wall, the wheels and feet together supporting the filtration unit off a ground surface of the enclosed space in use.

15. A disinfectant fogger and filtration device according to claims 12, wherein the side walls of the filtration housing comprise first and second spaced side walls, the first side wall comprising the first air opening, the second side wall comprising a second air opening.

16. A disinfectant fogger and filtration device according to claim 15, wherein the filtration device comprises a second filter disposed adjacent the second opening.

17. A disinfectant fogger and filtration device according to claim 16, wherein the first and second filters are disposed outside the filter housing.

18. A disinfectant fogger and filtration device according to claim 17, wherein the filter unit comprises first and second pockets mounted to the filter housing, the first pocket being configured to support the first filter and the second pocket being configured to support the second filter.

19. A disinfectant fogger and filtration device according to claim 12, wherein the filter housing comprises first and second mounting rails disposed on the top wall of the filter housing, the mounting rails being configured to support the fogger unit.

20. A disinfectant fogger and filtration device according to claim 19, wherein the first and second mounting rails are spaced from one another such that the fogger unit is able to be mounted between the mounting rails.

21-26. (canceled)
